(19) United States
(12) Patent Application Publication
(10) Pub. No.: US 2016/0125598 A1
(43) Pub. Date: May 5, 2016

(54) APPARATUS AND METHOD FOR AUTOMATED DETECTION OF LUNG CANCER

(71) Applicant: M.T.R TARGET LTD, Netanya (IL)

(72) Inventor: Iliya KUSNER, Haifa (IL)

(21) Appl. No.: 14/896,386

(22) PCT Filed: May 25, 2014
(86) PCT No.: PCT/IL2014/050468

§ 371 Date: Dec. 6, 2015

Related U.S. Application Data
(60) Provisional application No. 61/832,864, filed on Jun. 9, 2013.

(51) Int. Cl. G06T 7/00; G06K 9/52; A61B 6/00; G06K 9/62; A61B 6/03; G06T 7/20; G06T 7/60

(52) U.S. Cl. CPC G06T 7/0012; G06T 7/20; G06K 9/52; G06T 7/60; G06K 9/6267; A61B 6/032; A61B 6/50; G06T 2207/10072; G06K 2009/4666

(57) ABSTRACT

A method of computer aided detection of cancerous nodules of lung tissue, the method comprising: loading a plural-ity of contiguous tomographic images of a target area having a common axis; automatically detecting an object of interest in one of the loaded tomographic images of the target area; tracking the relative motion of the detected object of interest in adjacent ones of the loaded contiguous tomographic images; and identifying a potential lung cancer nodule responsive to the tracking.

### TECHNICAL FIELD

The present application relates to the field of automated diagnosis and decision support for medical imaging, and in particular to computer aided diagnosis (CAD) for lung cancer responsive to an analysis of a plurality of contiguous tomographic images.

### BACKGROUND OF THE INVENTION

Lung cancer is the most common diagnosed cancer around the world, accounting for over 1 million new cases annually, with a mortality rate in excess of 50%. Lung cancer prognosis varies greatly depending on the stage of detection, with early detecting resulting in a greatly improved prognosis.

Modern imaging, including various forms of computed tomography (CT), generates vast quantities of data. The term CT, defines a procedure that utilizes computer-processed rays to produce tomographic images, or ‘slices’, of specific areas of the patient body. These cross-sectional images are typically used for diagnostic and therapeutic purposes in various medical disciplines. Digital geometry processing is used to generate a three-dimensional image from a large series of two-dimensional X-ray images taken around a single axis of rotation. It would be advantageous to provide early detection of lung cancer based on such imaging, however the required manpower hours needed to search through the imaging data makes this a cost prohibitive process. The term CT is particularly meant to include by low dosage CT, positive emission tomography (PET) and a combination of PET and X-ray based CT, without limitation.

CAD for lung cancer is known to the prior art, and is typically based at least partially on image processing. In a first stage the lung tissue is identified from the surrounding tissue, and then utilizing image recognition nodules are identified.

The nodules are then analyzed using various algorithms, one example of which is described in U.S. Pat. No. 7,305,111 issued Dec. 4, 2007 to Arimura et al, the entire contents of which are incorporated herein by reference, to identify those which are indicative of cancer. The identified cancerous nodules are then reviewed by medical professionals to determine if action is to be performed.

One study has indicated that the percentage of false positive results from a single CT screening was in excess of 20%. After 2 rounds of CT screen the false positive rate was 33%. CAD has the potential to reduce the percentage of false positives, which generate needless expense and concern. The challenge faced by CAD for lung cancer is the particular difficulty in separating nodules from blood vessels, which are not distinguished by color or grey scale in the CT image. One of the main causes for false positive results in prior art CAD is the classification of a blood vessel as a nodule.

Certain CAD approaches have focused on providing filters to identify specific conditions, such as a particular filter designed to identify blood vessel bifurcation from nodules, however such an approach has not successfully brought CAD for CT lung cancer screening to an acceptable low false positive rate.

What is needed is a method of identifying nodules for further follow up, which successfully distinguishes the nodules from blood vessels.

### SUMMARY

Accordingly, it is a principal object of the present embodiments to overcome at least some of the disadvantages of the prior art. This is provided in certain embodiments by a method of computer aided detection of cancerous nodules of lung tissue, the method comprising: loading a plurality of contiguous tomographic images of a target area having a common axis; automatically detecting an object of interest in one of the loaded tomographic images of the target area; tracking the relative motion of the detected object of interest in adjacent ones of the loaded contiguous tomographic images; and identifying a potential lung cancer nodule responsive to the tracking.

Certain embodiments herein enable a method of computer aided detection of cancerous nodules of lung tissue, the method comprising: loading a plurality of contiguous tomographic images of a target area having a common axis; automatically detecting an object of interest in one of the loaded tomographic images of the target area; tracking the relative motion of the detected object of interest in adjacent ones of the loaded contiguous tomographic images; and identifying a potential lung cancer nodule responsive to the tracking.

In one embodiment the identifying comprises: in the event that the detected object of interest is stationary over a plurality of contiguous tomographic images, identifying the detected object of interest as the potential lung cancer nodule.

In another embodiment the identifying comprises: in the event that the detected object of interest does not appear in an adjacent one of the loaded contiguous tomographic images, identifying the detected object of interest as the potential lung cancer nodule.

In one embodiment the identifying comprises: in the event that the detected object of interest is of a size and trend axially inappropriate for a blood vessel, identifying the detected object of interest as the potential lung cancer nodule. In another embodiment the identifying comprises: in the event that the detected object of interest is detected as moving over a plurality of contiguous tomographic images, identifying the detected object of interest as not being a potential lung cancer nodule

In one embodiment the identifying comprises: in the event that the detected object of interest is detected as branching over a plurality of contiguous tomographic images, identifying the detected object of interest as not being a potential lung cancer nodule. In another embodiment the identifying comprises: determining the size, axial location and trend of the detected object of interest; and in the event that the determined size, axial location and trend of the detected object of interest do not meet a predetermined definition for blood vessels, identifying the detected object of interest as the potential lung cancer nodule.

Independently, certain embodiments herein enable a system arranged to detect cancerous nodules of lung tissue, the system comprising: a processor; and a display, the processor arranged to: load a plurality of contiguous tomographic images of a target area having a common axis; detect an object of interest in one of the loaded tomographic images of the target area; track the relative motion of the detected object of interest in adjacent ones of the loaded contiguous tomographic images; and identify a potential lung cancer nodule responsive to the tracking.

In one embodiment the identifying by the processor comprises: in the event that the detected object of interest is stationary over a plurality of contiguous tomographic images, identify the detected object of interest as the potential lung cancer nodule. In another embodiment the identifying by the processor comprises: in the event that the detected object of interest does not appear in an adjacent one of the loaded contiguous tomographic images, identify the detected object of interest as the potential lung cancer nodule.

In one embodiment the identifying by the processor comprises: in the event that the detected object of interest is of a size and trend axially inappropriate for a blood vessel, identify the detected object of interest as the potential lung cancer nodule. In another embodiment the identifying by the processor comprises: in the event that the detected object of interest is detected as moving over a plurality of contiguous tomographic images, identify the detected object of interest as not being a potential lung cancer nodule.

In one embodiment the identifying by the processor comprises: in the event that the detected object of interest is detected as branching over a plurality of contiguous tomographic images, identify the detected object of interest as not being a potential lung cancer nodule. In another embodiment the identifying by the processor comprises: determine the size, axial location and trend of the detected object of interest; and in the event that the determined size, axial location and trend of the detected object of interest do not meet a predetermined definition for blood vessels, identify the detected object of interest as the potential lung cancer nodule.

Independently, certain embodiments herein enable a non-transitory computer readable medium containing computer readable instructions, the computer readable instructions arranged to control a processor to perform a method of computer aided detection of cancerous nodules of lung tissue, the method comprising: loading a plurality of contiguous tomographic images of a target area having a common axis; automatically detecting an object of interest in one of the loaded tomographic images of the target area; tracking the relative motion of the detected object of interest in adjacent ones of the loaded contiguous tomographic images; and identifying a potential lung cancer nodule responsive to the tracking.

In one embodiment the identifying of the method comprises: in the event that the detected object of interest is stationary over a plurality of contiguous tomographic images, identifying the detected object of interest as the potential lung cancer nodule. In another embodiment the identifying of the method comprises: in the event that the detected object of interest does not appear in an adjacent one of the loaded contiguous tomographic images, identifying the detected object of interest as the potential lung cancer nodule.

In one embodiment the identifying of the method comprises: in the event that the detected object of interest is of a size and trend axially inappropriate for a blood vessel, identifying the detected object of interest as the potential lung cancer nodule. In another embodiment the identifying of the method comprises: in the event that the detected object of interest is detected as moving over a plurality of contiguous tomographic images, identifying the detected object of interest as not being a potential lung cancer nodule.

In one embodiment the identifying of the method comprises: in the event that the detected object of interest is detected as branching over a plurality of contiguous tomographic images, identifying the detected object of interest as not being a potential lung cancer nodule. In another embodiment the identifying of the method comprises: determining the size, axial location and trend of the detected object of interest; and in the event that the determined size, axial location and trend of the detected object of interest do not meet a predetermined definition for blood vessels, identifying the detected object of interest as the potential lung cancer nodule.

Additional features and advantages of the invention will become apparent from the following drawings and description.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 illustrates a high level block diagram of a system arranged to computer aided detection of cancerous nodules of lung tissue in cooperation with an imager;

FIG. 2 illustrates a high level flow chart of an exemplary embodiment of a method of computer aided detection of cancerous nodules of lung tissue for use with the system of FIG. 1;

FIG. 3 illustrates a first and a second object of interest in adjacent tomographic images; and

FIG. 4 illustrates a high level flow chart of an exemplary embodiment of a more detailed method of computer aided detection of cancerous nodules of lung tissue for use with the system of FIG. 1.

### DETAILED DESCRIPTION OF PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

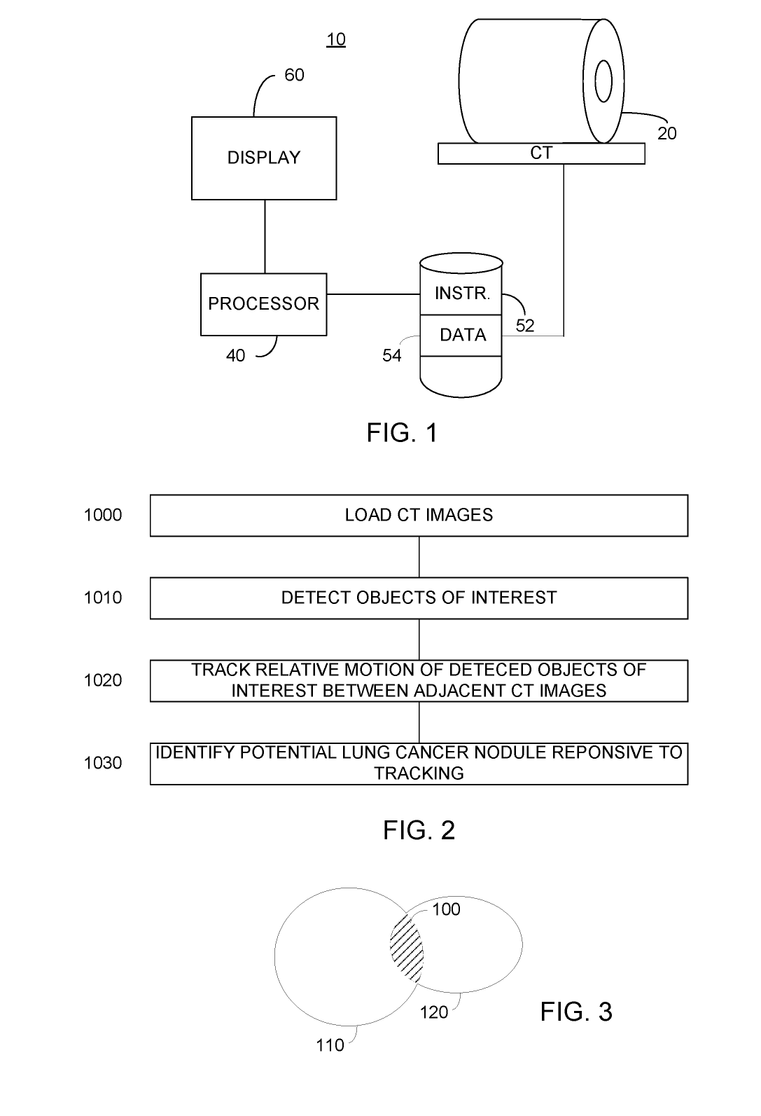

FIG. 1 illustrates a high level block diagram of a system 10 arranged to computer aided detection of cancerous nodules of lung tissue in cooperation with an imager 20, imager 20 illustrated without limitation as a CT device. System 10 comprises a processor 40 in communication with a memory 50 and a display 60. Memory 50 comprises an instruction portion 52 and a data portion 54. Memory 50 may be dedicated to processor 40, such as in a computer or workstation. Alternatively, all, or part of memory 50 may be shared with processor 40 such as in a cloud computing or networked environment, without limitation. Display 60 may similarly be dedicated to processor 40, or shared among a plurality of processors 40 without limitation.

The operation of system 10 will be described further below in detail relation to FIGS. 2 and 3 below, however in general instruction portion 52 of memory 50 stores non-transitory computer readable instructions. Processor 40 is arranged to read the stored non-transitory computer readable instructions from instruction portion 52 and in accordance therewith perform the below described method of computer aided detection of cancerous nodules of lung tissue. Data, such as CT images of patient body, may be loaded from imager 20 and stored on data portion 54. Alternately, data portion 54 is not supplied, and CT images of the patient body are loaded from imager 20.

FIG. 2 illustrates a high level flow chart of an exemplary embodiment of a method of computer aided detection of cancerous nodules of lung tissue for use with system 10. In stage 1000, a plurality of contiguous CT images of a patient body are loaded into processor 40 from data portion 52, or from imager 20. In stage 1010, objects of interest (OOI) are detected in the various loaded contiguous CT images of stage 1000. In an exemplary embodiment, OOI are identified by their grey scale level in relation to the surrounding tissue in the images. Preferably, each OOI is identified utilizing known algorithms which remove unwanted noise from the identification. Each OOI is preferably provided with an identifier associated therewith.

In stage 1020, for each of OOI of stage 1010, the adjacent slices are reviewed to track the progression of the OOI. In particular, the location of the OOI in the first selected tomographic image is determined, as well as its external contour and optionally any defined shapes identified within the external contour. For each adjacent tomographic image, the same location is reviewed to determine if the OOI is present, and thereby determine in an OOI in the adjacent tomographic image is a continuation of the particular OOI. An intersection of greater than a predetermined percentage, which in one embodiment is 20%, is used to determine that an OOI in the adjacent tomographic image is a continuation of the particular OOI. In such an event the identifiers of the OOI are set as a single identifier.

Referring to FIG. 3, an intersection 100 between an OOI 110 of first tomographic image and an OOI 120 of a second tomographic image, adjacent the first tomographic image is illustrated. As described above, in one embodiment in the event that the area of intersection 100 is at least 20% of the area of OOI 110, OOI 120 will be determined to be a continuation of OOI 110.

The above has been described in an embodiment wherein intersection of greater than a predetermined amount is exclusively utilized to determine continuation of an OOI, however this is not meant to be limiting in any way. In another embodiment the contour of the OOI and the contour of the adjacent OOI are taken into account to determine if the adjacent OOI is a continuation of the present OOI or is an independent OOI. In yet another embodiment one or more of: the external contour; any detected internal shapes; and the total area of intersection 100 are further taken into account in the determination if the adjacent OOI is a continuation of the present OOI or is an independent OOI. In yet another embodiment the speed of movement is further taken into account, with the speed of movement defined as the distance between locations of OOIs divided by the number of tomographic images over which the distance is experienced. In particular, the distance per tomographic image of the OOI from one tomographic image to an adjacent tomographic image is taken into account to determine if the adjacent OOI is a continuation of the present OOI or is an independent OOI.

Stage 1020 is thus accomplished or all detected OOIs of stage 1010, including the determination of which OOIs are continuation of an OOI from an adjacent tomographic image.

In stage 1030, potential lung cancer nodules are identified responsive to the tracking of stage 1020. In particular, and as will be described further below, nodules are identified by being stationary objects in position across a plurality of tomographic images, as opposed to blood vessels which tend to meander across locations of adjacent tomographic images. Blood vessels similarly tend to be continuous and do not disappear between adjacent tomographic images. Blood vessels exhibit branching which is not normally associated with a cancerous nodule. Additionally, an OOI may be identified as a non-blood vessel due to its size and trend in relation to the lung. In particular, blood vessels tend to diminish in diameter as they extend further from the lung center. Thus if an OOI exhibits a diameter greater than a normal diameter for a blood vessel the present location in relation to an axis center, which represents near a lung center, it can be identified as a potentially cancerous nodule. Additionally, if an OOI does not diminish is diameter, as it continues to extend axially from the lung center, it can be identified as a potentially cancerous nodule.

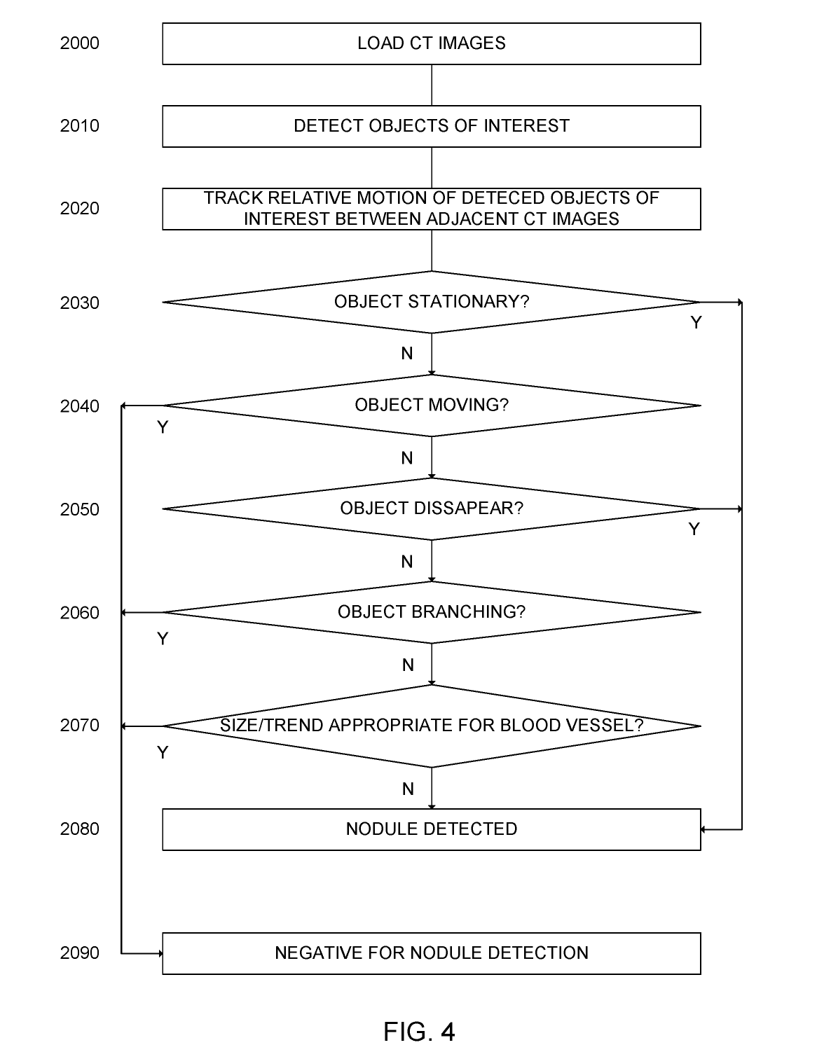

FIG. 4 illustrates a high level flow chart of an exemplary embodiment of a more detailed method of computer aided detection of cancerous nodules of lung tissue for use with system 10. In stage 2000, a plurality of contiguous CT images of a patient body are loaded into processor 40 from data portion 52, or from imager 20. In stage 2010, OOIs are detected in the various loaded contiguous CT images of stage 2000. In an exemplary embodiment, OOI are identified by their coloring in relation to the surrounding tissue in the images. Preferably, each OOI is identified utilizing known algorithms which remove unwanted noise from the identification. Each 00I is preferably provided with an identifier associated therewith.

In stage 2020, for each of OOI of stage 2010, the adjacent slices are reviewed to track the progression of the OOI as described above in relation to stage 1020. Stage 2020 is thus accomplished or all detected OOIs of stage 2010, including the determination of which OOIs are continuation of an OOI from an adjacent tomographic image.

In stage 2030, it is determined if the selected OOI is stationary over the adjacent tomographic images. Preferably, the location is determined as relatively constant over a plurality of adjacent tomographic images, i.e. the OOI is determined as stationary if the overlap 100 between adjacent images 110, 120 is higher than a given threshold. In further detail, in certain embodiments the determination that the selected OOI is stationary is determined responsive to one or more of: the degree of OOI overlap as described above; the external contour of the OOI in relation to the external contour of the adjacent OOI; the appearance of any detected internal shapes of the subject OOI in the adjacent OOI; the total area of the overlap between the subject OOI and the OOI of the adjacent tomographic image; and the speed of movement of the subject OOI, as described above. In one embodiment, a score of the factors is utilized to determine if the OOI is stationary. In the event that it is determined that the selected OOI is stationary over the adjacent tomographic images, in stage 2080 it is determined that a potentially cancerous nodule is detected. Preferably, a marking of the location, and detailed location information is output to display 60, and further preferably stored on memory 50, particularly on memory portion 54.

In stage 2040, it is determined if the selected OOI moves over the adjacent tomographic images. Preferably, the definition of moving is not identical with the opposite of stationary of stage 2030, and is indicative only when the selected OOI meanders over a plurality of adjacent tomographic images such that the OOI is axially removed from the original selected OOI location by more than a predetermined minimum after a predetermined number of tomographic images. In the event that it is determined that the selected OOI is determined as moving over the adjacent tomographic images, in stage 2090 it is determined that the selected OOI is not a potential lung cancer nodule to be further investigated. Detection of the selected OOI as a non-nodule is preferably stored on memory 50, particularly on memory portion 54.

In stage 2050, it is determined if the selected OOI fails to appear in an adjacent tomographic image in any form. Such an event, also known as a disappearance, is indicative of a potentially cancerous nodule, and not that of a blood vessel, which as described above generally meanders over a plurality of adjacent tomographic images. In further detail the determination that the selected OOI had disappeared is determined responsive to one or more of: the external contour of the selected OOI, and the lack of a congruent OOI in the adjacent tomographic image; the disappearance of any detected internal shapes of the selected OOI in an OOI of the adjacent tomographic image; the total area of intersection 100 between the selected OOI and any OOI in the adjacent image being less than a predetermined amount, which in one non-limiting embodiment is 20%; and the speed of movement of the selected OOI being greater than a predetermined maximum. In one embodiment, a score of the factors is utilized to determine if the selected OOI has disappeared. In the event that the selected OOI fails to appear in an adjacent tomographic image, stage 2080 as described above is performed.

In stage 2060 it is determined if there is branching of the OOI, which is characteristic of blood vessels and not of nodule. The determination of branching is determined responsive to one or more of: the degree of OOI overlap as described above, external contour of the OOI in relation to the external contour of OOI of the adjacent tomographic image; the appearance of any detected internal shapes of the subject OOI in the adjacent OOI; the total area of the overlap between the subject OOI and the OOI of the adjacent tomographic image; and the speed of movement of the subject OOI, as described above. In one embodiment, a score of the factors is utilized to determine if the OOI is branching. For example, in the event that in an adjacent tomographic image a plurality of OOIs appear with intersection less than the predetermined intersection cut-off, described above in a non-limiting example as 20%, stage 2060 determines that branching of the original OOI has occurred. Since such branching is associated with a blood vessel, and not with a potential lung cancer nodule, stage 2090 as described above is performed for the present OOI.

In stage 2070, the OOI is analyzed to determine if it is appropriate to be determined as a blood vessel. In particular, the size of the OOI is analyzed in relation to location in the lung. It is known that blood vessels are larger close to the trachea, which is itself typically near the tomographic image axis, and become smaller towards the peripheral. Predetermined values of normal structure are in one embodiment stored in data portion 54, and in another embodiment instructions to calculate the values are stored in instruction portion 52. Processor 40 thus determines if the detected OOI is appropriate for a blood vessel, responsive to its size and location. Similarly, an OOI which does not trend to reduce in size as it extends further from the trachea location, in accordance with stored algorithm or stored data points, is indicative of a blood vessel.

In the event that the size and/or trend of the OOI is not indicative of a blood vessel, stage 2080 is performed, and in the event that the the size and/or trend of the OOI is indicate of a possible blood vessel stage 2090 is performed.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof which would occur to persons skilled in the art upon reading the foregoing description and which are not in the prior art.

1. A method of computer aided detection of cancerous nodules of lung tissue, the method comprising:
loading a plurality of contiguous tomographic images of a target area having a common axis;
automatically detecting an object of interest in one of the loaded tomographic images of the target area;
tracking the relative motion of the detected object of interest in adjacent ones of said loaded contiguous tomographic images; and
identifying a potential lung cancer nodule responsive to said tracking.

2. The method according to claim 1, wherein said identifying comprises:
in the event that the detected object of interest is stationary over a plurality of contiguous tomographic images, identifying the detected object of interest as the potential lung cancer nodule.

3. The method according to claim 1, wherein said identifying comprises:
in the event that the detected object of interest does not appear in an adjacent one of said loaded contiguous tomographic images, identifying the detected object of interest as the potential lung cancer nodule.

4. The method according to claim 1, wherein said identifying comprises:
in the event that the detected object of interest is of a size and trend axially inappropriate for a blood vessel, identifying the detected object of interest as the potential lung cancer nodule.

5. The method according to claim 1, wherein said identifying comprises:
in the event that the detected object of interest is detected as moving over a plurality of contiguous tomographic images, identifying the detected object of interest as not being a potential lung cancer nodule

6. The method according to claim 1, wherein said identifying comprises:
in the event that the detected object of interest is detected as branching over a plurality of contiguous tomographic images, identifying the detected object of interest as not being a potential lung cancer nodule.

7. The method according to claim 1, wherein said identifying comprises:
determining the size, axial location and trend of the detected object of interest; and
in the event that the determined size, axial location and trend of the detected object of interest do not meet a predetermined definition for blood vessels, identifying the detected object of interest as the potential lung cancer nodule.

8. A system arranged to detect cancerous nodules of lung tissue, the system comprising:
a processor; and
a display, said processor arranged to:
load a plurality of contiguous tomographic images of a target area having a common axis;
detect an object of interest in one of the loaded tomographic images of the target area;
track the relative motion of the detected object of interest in adjacent ones of said loaded contiguous tomographic images; and
identify a potential lung cancer nodule responsive to said tracking.

9. The system according to claim 8, wherein said identifying by said processor comprises:
in the event that the detected object of interest is stationary over a plurality of contiguous tomographic images, identify the detected object of interest as the potential lung cancer nodule.

10. The system according to claim 8, wherein said identifying by said processor comprises:
in the event that the detected object of interest does not appear in an adjacent one of said loaded contiguous tomographic images, identify the detected object of interest as the potential lung cancer nodule.

11. The system according to claim 8, wherein said identifying by said processor comprises:
in the event that the detected object of interest is of a size and trend axially inappropriate for a blood vessel, identify the detected object of interest as the potential lung cancer nodule.

12. The system according to claim 8, wherein said identifying by said processor comprises:
in the event that the detected object of interest is detected as moving over a plurality of contiguous tomographic images, identify the detected object of interest as not being a potential lung cancer nodule.

13. The system according to claim 8, wherein said identifying by said processor comprises:
in the event that the detected object of interest is detected as branching over a plurality of contiguous tomographic images, identify the detected object of interest as not being a potential lung cancer nodule.

14. The system according to claim 8, wherein said identifying by said processor comprises:
determine the size, axial location and trend of the detected object of interest; and
in the event that the determined size, axial location and trend of the detected object of interest do not meet a predetermined definition for blood vessels, identify the detected object of interest as the potential lung cancer nodule.

15. A non-transitory computer readable medium containing computer readable instructions, the computer readable instructions arranged to control a processor to perform a method of computer aided detection of cancerous nodules of lung tissue, the method comprising:
loading a plurality of contiguous tomographic images of a target area having a common axis;
automatically detecting an object of interest in one of the loaded tomographic images of the target area;
tracking the relative motion of the detected object of interest in adjacent ones of said loaded contiguous tomographic images; and
identifying a potential lung cancer nodule responsive to said tracking.

16. The non-transitory computer readable medium according to claim 15, wherein said identifying of the method comprises:
in the event that the detected object of interest is stationary over a plurality of contiguous tomographic images, identifying the detected object of interest as the potential lung cancer nodule.

17. The non-transitory computer readable medium according to either claim 15, wherein said identifying of the method comprises:
in the event that the detected object of interest does not appear in an adjacent one of said loaded contiguous tomographic images, identifying the detected object of interest as the potential lung cancer nodule.

18. The non-transitory computer readable medium according to claim 15, wherein said identifying of the method comprises:
in the event that the detected object of interest is of a size and trend axially inappropriate for a blood vessel, identifying the detected object of interest as the potential lung cancer nodule.

19. The non-transitory computer readable medium according to claim 15, wherein said identifying of the method comprises:
in the event that the detected object of interest is detected as moving over a plurality of contiguous tomographic images, identifying the detected object of interest as not being a potential lung cancer nodule.

20. The non-transitory computer readable medium according to claim 15, wherein said identifying of the method comprises:
in the event that the detected object of interest is detected as branching over a plurality of contiguous tomographic images, identifying the detected object of interest as not being a potential lung cancer nodule.

21. The non-transitory computer readable medium according to claim 15, wherein said identifying of the method comprises:
determining the size, axial location and trend of the detected object of interest; and
in the event that the determined size, axial location and trend of the detected object of interest do not meet a predetermined definition for blood vessels, identifying the detected object of interest as the potential lung cancer nodule.
